(19) United States
(12) Patent Application Publication
(10) Pub. No.: US 2011/0071434 A1
(43) Pub. Date: Mar. 24, 2011

(54) Apparatus for Collecting Urine and Other Bodily Fluids

(76) Inventor: Jacqueline E. Higgins, Poughkeepsie, NY (US)

(21) Appl. No.: 12/564,757

(22) Filed: Sep. 22, 2009

(51) Int. Cl. A61B 5/20

(52) U.S. Cl. 600/573

(57) ABSTRACT

An apparatus for collecting urine and other bodily fluids comprises an elongate handle and a specimen collection cup. The elongate handle defines an opening therein and comprises a selectively detachable portion. The selectively detachable portion at least partially forms the perimeter edge of the opening. The specimen collection cup is at least partially disposed within the opening in the elongate handle and is removably supported by the elongate handle.

### FIELD OF THE INVENTION

The present invention relates generally to medical devices, and, more particularly, to apparatus for collecting body fluids like urine for purposes of medical screening and diagnostics.

### BACKGROUND OF THE INVENTION

In the medical setting, the collection of urine samples is a very common occurrence. Urinalysis is used, for example, as a screening and diagnostic tool to help detect substances or cellular material in the urine associated with several different metabolic and kidney disorders. It is also used to detect urinary tract infections and other disorders of the urinary tract, as well as to detect illegal drug use. Finally, in patients with acute or chronic conditions, such as kidney disease, urinalysis may be ordered as a rapid and inexpensive means of monitoring organ function, status, and response to treatment.

For this reason, the collection of urine samples is a vitally important step in the health care setting and must be performed carefully. When taking urine samples, for example, it is usually necessary to maintain the sterility of the rim and inside of the specimen collection cup (i.e., specimen collection container) while, at the same time, providing the patient with an ergonomically friendly device and collection method. Unfortunately, commonly used urine collection devices, especially those used by women, are generally not ergonomically friendly, making it difficult for women to collect their urine in a comfortable and sterile manner. Moreover, it is even more difficult for morbidly obese men and women, elderly people, children, and women at the later stages of pregnancy to collect their own urine sample in a sanitary and hygienic manner due impediments to movement inherent to their age or condition.

Information relevant to attempts to address these problems can be found in, for example, U.S. Pat. Nos. 5,147,342 to Kane et al., and 6,485,438 to Minue; and in U.S. Patent Application Nos. 2002/0169395 to Huang, 2008/0077047 to Karvas et al., and 2009/0118642 to Washington, each of which utilizes a handle arrangement to aid in the collection of urine samples. However, each of these references suffers from one or more of the following disadvantages: high cost of manufacture, high complexity of manufacture, difficulty in use, non-disposability (and the associated danger of specimen contamination), and inability to accommodate specimen collection cups of differing sizes.

For the foregoing reasons, there is a need for apparatus that are disposable, inexpensive, easy to manufacture, and allow urine to be collected by a donor in an ergonomic and hygienic fashion while maintaining sample integrity. These apparatus will also preferably be able to accommodate specimen collection cups of differing sizes.

### SUMMARY OF THE INVENTION

Embodiments of the present invention address the above-identified need by providing apparatus that allow urine and other bodily fluids to be collected in an ergonomic and hygienic fashion while maintaining sample integrity.

In accordance with an aspect of the invention, an apparatus for collecting urine and other bodily fluids comprises an elongate handle and a specimen collection cup. The elongate handle defines an opening therein and comprises a selectively detachable portion. The selectively detachable portion at least partially forms the perimeter edge of the opening. The specimen collection cup is at least partially disposed within the opening in the elongate handle and is removably supported by the elongate handle.

In accordance with one of the above-identified embodiments of the invention, an apparatus for collecting urine specimens comprises an elongate handle that has a length substantially greater than its width. The elongate handle defines an opening proximate to one of its lengthwise ends and is adapted to support a specimen collection cup by having the cup inserted partially into the opening. To accommodate specimen collection cups of differing sizes, the elongate handle comprises a detachable portion that forms a portion of the perimeter of the opening. The detachable portion is attached to the remainder of the elongate handle along a series of perforations, allowing the detachable portion to be selectively removed by hand without the use of tools. With the detachable portion in place, the opening is of a first size, and when the detachable portion is removed, the opening of a second larger size.

Advantageously, the above described embodiment allows the donor patient to collect a urine sample in an ergonomic and hygienic manner. The patient donor need only grasp the elongate handle at its lengthwise end opposite the specimen collection cup, and then utilize the reach of the elongate handle to comfortably position the specimen collection cup into that patient's stream of urine. The patient donor does not need to handle the specimen collection cup directly, and cleanliness and specimen integrity (i.e., non-contamination) are thereby maintained.

### BRIEF DESCRIPTION OF THE DRAWINGS

These and other features, aspects, and advantages of the present invention will become better understood with regard to the following description, appended claims, and accompanying drawings where:

FIG. 1A shows a perspective view of an apparatus in accordance with a first illustrative embodiment of the invention;

FIG. 1B shows a plan view of the FIG. 1A apparatus;

FIG. 1C shows a side view of the FIG. 1A apparatus;

FIG. 2 shows a plan view of an apparatus in accordance with a second illustrative embodiment of the invention;

FIG. 3 shows a plan view of an apparatus in accordance with a third illustrative embodiment of the invention;

FIG. 4 shows a plan view of an apparatus in accordance with a fourth illustrative embodiment of the invention; and

FIG. 5 shows a plan view of an apparatus in accordance with a fifth illustrative embodiment of the invention.

### DETAILED DESCRIPTION OF THE INVENTION

The present invention will be described with reference to illustrative embodiments. For this reason, numerous modifications can be made to these embodiments and the results will still come within the scope of the invention. No limitations with respect to the specific embodiments described herein are intended or should be inferred.

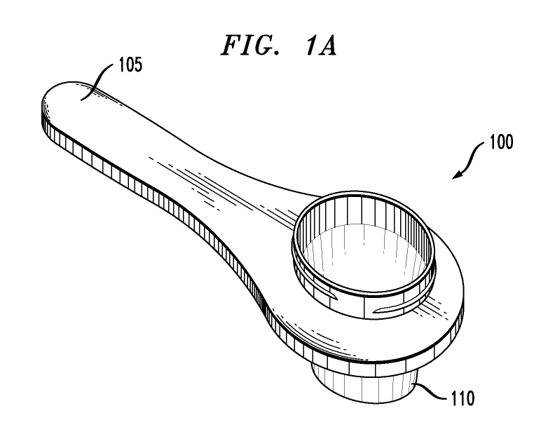

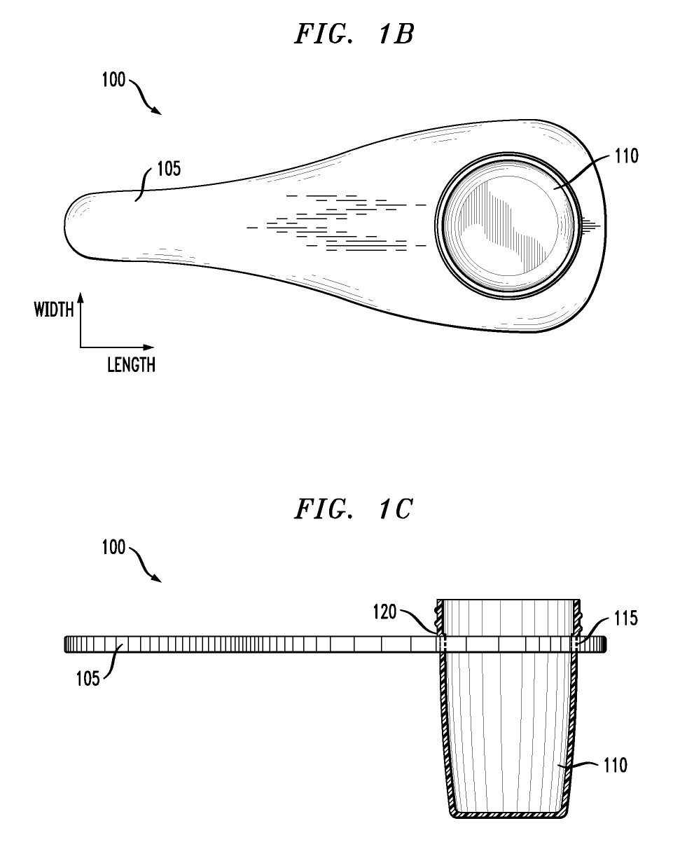

FIGS. 1A-1C show a perspective view, a plan view, and a side view, respectively, of an apparatus 100 in accordance with a first illustrative embodiment of the invention. The apparatus comprises an elongate handle 105 and specimen collection cup 110. The elongate handle has a length that is substantially greater than its maximum width and defines an opening 115 proximate to one of its lengthwise ends. When considered top-down as shown in FIG. 1B, for example, the elongate handle is approximately bottle shaped, gradually tapering from a smaller width to larger width along its length. The opening is disposed in the widest portion of this “bottle.”

The opening 115 provides the mechanism by which the elongate handle 105 supports the specimen collection cup 110. More particularly, the opening in the elongate handle is dimensioned so as to removably support the specimen collection cup by having all or a portion of the edge of the opening contact the specimen collection cup after the specimen collection cup is inserted sufficiently therein. In the arrangement shown in FIGS. 1A-1C, the specimen collection cup comprises an external lip 120 which rests on the uppermost edge of the opening in the elongate handle when the specimen collection cup is partially inserted into the opening. However, if, instead, the specimen collection cup were tapered and did not have such a lip, it could be supported by having the opening in the elongate handle be dimensioned such that all or a portion of the opening's edge makes contact with the progressively widening sidewalls of the specimen collection cup at some point along the cup's height. In either case, once so supported, the specimen collection cup is held in place by gravity against the edge of the opening in the elongate handle so long as the elongate handle and specimen collection cup are oriented substantially upward relative to the Earth. Removal of the specimen collection cup from the elongate handle after a specimen is collected is then only a matter of sliding the specimen collection cup upward out of the opening.

Notably, the apparatus 100 provides a donor patient with a number of advantages. It, for example, allows the donor patient to collect a urine sample in an ergonomic and hygienic manner. The patient donor need only grasp the elongate handle 105 at its lengthwise end opposite the specimen collection cup 110, and then utilize the reach of the elongate handle to comfortably position the specimen collection cup into that patient's stream of urine. The patient donor does not need to handle the specimen collection cup directly, and cleanliness and specimen integrity (i.e., non-contamination) are thereby maintained.

To further improve cleanliness and reduce the possibility of contamination, moreover, the elongate handle 105 will preferably be disposable. The elongate handle may, for example, comprise plastic, wood, paper, or some combination thereof. These materials are generally easy to manufacture into the shape of the illustrative elongate handle, inexpensive, readily available, and may be formed with the necessary rigidity to support most conventional specimen collection cups when filled.

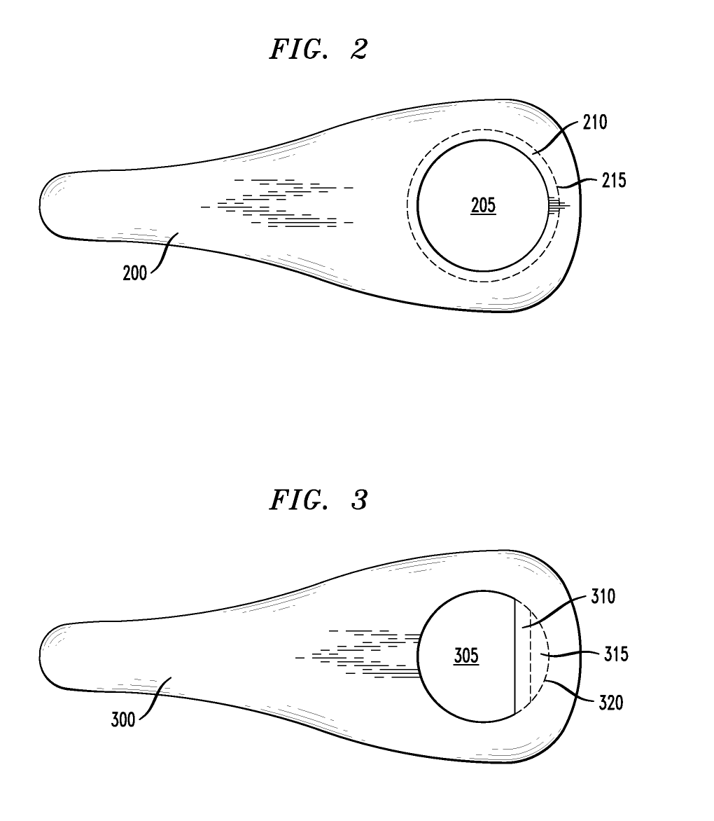

Nevertheless, it will be appreciated that specimen collection cups, unlike the elongate handle 105, are commercially available and are produced in a variety of shapes and sizes. A survey of commercially available specimen collection cups with specimen volumes between 40 milliliters (mL) and 180 mL, for example, revealed specimen collection cups with uppermost rim diameters that varied from 30 millimeters (mm) to 60 mm. Moreover, as briefly mentioned above, specimen collection cups may be tapered or straight walled. As a result, it is preferable to have elongate handles that are individually capable of accommodating specimen collection cups of differing sizes. FIGS. 2-5 show elongate handle embodiments that provide this added adaptability.

FIG. 2, for example, shows a plan view of an elongate handle 200 in accordance with a second illustrative embodiment of the invention. As before, the elongate handle defines an opening 205 proximate to one of its lengthwise ends. However, unlike the handle shown in FIGS. 1A-1C, this elongate handle also comprises a detachable portion 210. The detachable portion forms the entire perimeter of the opening and is attached to the remainder of the elongate handle along a series of perforations 215 in the elongate handle. These perforations, in turn, allow the detachable portion to be selectively detached (i.e., removed) from the remainder of the elongate handle by hand without the use of tools (e.g., by tearing it off along the perforations). Doing so substantially expands the size of the opening. In this manner, the elongate handle can be used to support a specimen collection cup of one size with the detachable portion in place, and to support a specimen collection cup of a larger size with the detachable portion removed. In other words, the user has a choice between two different opening sizes.

Likewise, FIG. 3 shows an elongate handle 300 having an expandable opening 305, this elongate handle in accordance with a third illustrative embodiment of the invention. This elongate handle includes two detachable portions, namely a first detachable portion 310 and a second detachable portion 315, both of which are attached to each other and the remainder of the elongate handle by a series of perforations 320. While in place, the first detachable portion forms a portion of the perimeter edge of the opening. As a result, selectively detaching the first portion expands the opening somewhat. Selectively detaching both the first and second portions, moreover, expands the opening even further. Accordingly, this arrangement gives the user the choice of three different opening sizes.

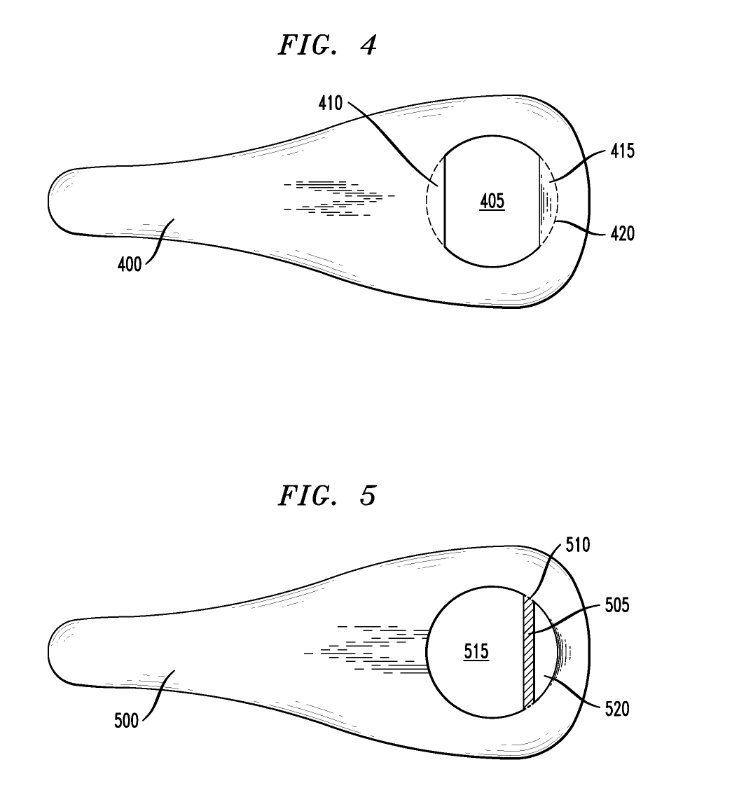

FIG. 4 shows a plan view of an elongate handle 400 with an opening 405, this one in accordance with a fourth illustrative embodiment of the invention. In this case, the elongate handle comprises a first detachable portion 410 and a second detachable portion 415 that are detachably attached to the remainder of the elongate handle at two opposing sides of the opening by a series of perforations 420. Each detachable portion, while in place, forms a portion of the perimeter of the opening. Selective detachment of the first or second detachable portion expands the opening to some extent. Selective detachment of both the first and second detachable portions expands the opening even further.

Lastly, FIG. 5 shows a plan view of an elongate handle 500 in accordance with a fifth illustrative embodiment of the invention. Here, a detachable portion 505 is again attached to the remainder of the elongate handle along a series of perforations 510 and forms a portion of the perimeter of, in this case, both a first opening 515 and second opening 520. Selective detachment of the detachable portion acts to merge the first and second openings, effectively forming a single larger opening. The single larger opening is able to accommodate a larger specimen collection cup than the original first opening.

As can be seen from the embodiments described in FIGS. 2-5, one or more selectively detachable portions can be integrated into an elongate handle to form an elongate handle that is operative to accommodate specimen collection cups of differing sizes. However, while several arrangements have been shown, these particular arrangements are only meant to be illustrative and are by no means intended to be comprehensive. Clearly, other embodiments can use different types and arrangements of elements for implementing the described functionality. One skilled in the art will recognize these numerous alternative embodiments within the scope of the appended claims.

Moreover, while the detachable portions are attached to the remainder of the elongate handle along a series of perforations in the above-described embodiments, other forms of attachment may be used and the resultant apparatus will still come within the scope of the invention. A detachable portion may be attached to the remainder of its elongate handle by, for example, an adhesive, double-sided tape, frangible tabs or pins, and by several other such mechanisms.

Lastly, all the features disclosed herein may be replaced by alternative features serving the same, equivalent, or similar purposes, unless expressly stated otherwise. Thus, unless expressly stated otherwise, each features disclosed is one example only of a generic series of equivalent or similar features.

1. (canceled)

2. The apparatus of claim 17, wherein detachment of the selectively detachable portion is operative to cause the opening to increase in size.

3. The apparatus of claim 17, wherein the selectively detachable portion forms the entire perimeter edge of the opening.

4. The apparatus of claim 17, wherein the selectively detachable portion forms less than the entire perimeter edge of the opening.

5. (canceled)

6. The apparatus of claim 17, wherein the elongate handle comprises an uppermost surface that is substantially flat.

7. The apparatus of claim 17, wherein the elongate handle comprises at least one of plastic, wood, and paper.

8. The apparatus of claim 17, wherein the selectively detachable portion is operative to be selectively detached from the remainder of the elongate handle by hand without the use of tools.

9. The apparatus of claim 17, wherein the selectively detachable portion is detachably attached to the remainder of the elongate handle along a series of perforations cut into the elongate handle.

10. The apparatus of claim 17, wherein the selectively detachable portion is detachably attached to the remainder of the elongate handle by at least one of an adhesive, tape, tabs, and pins.

11. The apparatus of claim 17, wherein the elongate handle defines an additional opening therein.

12. The apparatus of claim 17, wherein the elongate handle comprises an additional selectively removable portion.

13. (canceled)

14. (canceled)

15. The apparatus of claim 17, wherein at least a portion of the supported specimen collection cup contacts an edge of the opening.

16. The apparatus of claim 17, wherein at least a portion of the supported specimen collection cup contacts the selectively detachable portion.

17. An apparatus for collecting urine and other bodily fluids, the apparatus comprising:
an elongate handle, the elongate handle defining an opening therein which is substantially proximate to a lengthwise end of the elongate handle and comprising a selectively detachable portion, the selectively detachable portion at least partially forming a perimeter edge of the opening; and
a specimen collection cup, the specimen collection cup at least partially disposed within the opening in the elongate handle and removably supported by the elongate handle.

18. (canceled)

19. (canceled)
